(19) United States
(12) Patent Application Publication
(10) Pub. No.: US 2025/0368932 A1
(43) Pub. Date: Dec. 4, 2025

(54) SYSTEM FOR MIMICKING CELLULAR MICROENVIRONMENT AND METHOD FOR EVALUATING CELL DEVELOPMENT OR DRUG THERAPY EFFECTIVENESS USING THE SAME

(71) Applicant: Anivance AI Corporation, Hsinchu County (TW)

(72) Inventors: Guan-Yu Chen, Hsinchu County (TW); Chien-Yu Huang, Nantou County (TW); Yu-Chien Lin, Taipei City (TW)

(73) Assignee: National Yang Ming Chiao Tung University, Hsinchu City (TW)

(21) Appl. No.: 18/679,497

(22) Filed: May 31, 2024

(30) Foreign Application Priority Data
May 31, 2024 (TW) 113120351

(51) Int. Cl. C12M 3/00; C12M 1/12; C12M 1/34; C12M 1/42; G01N 33/50

(52) U.S. Cl. CPC C12M 21/08; C12M 25/14; C12M 35/02; C12M 41/46; G01N 33/5008

(57) ABSTRACT

A system for mimicking cellular microenvironment is provided and includes a chip, a hydrogel, and cells in contact with the hydrogel. The chip includes a first carrier having a pore and a second carrier coupled to the first carrier. The hydrogel is disposed in the pore and includes extracellular matrix. Also provided is a method for evaluating cell development, including providing the system for mimicking cellular microenvironment; providing a stimulation to a responsive hydrogel; and evaluating the cell development. Further provided is a method for evaluating drug therapy effectiveness, including providing the system for mimicking cellular microenvironment; administering the drug to the cells; and evaluating drug therapy effectiveness.

### BACKGROUND OF THE INVENTION

### 1. Field of the Invention

The present disclosure relates to methods for evaluating cell development and drug therapeutic effects, and in particular, to methods for evaluating cell development or drug therapy effectiveness by simulating cell microenvironments.

### 2. Description of the Prior Art

Tumor microenvironment consists of tumor cells and the extracellular matrix (ECM). Tumor cells alter their morphology and motility due to the dynamic mechanical properties of the microenvironment, such as matrix rigidity, stretching, compression, and shear stress. These factors contribute to the malignancy and treatment resistance of tumor cells. Therefore, cancer mechanobiology has emerged as a new field in cancer research. However, current cancer research models and preclinical in vitro drug testing focus on establishing static extracellular matrix or static tumor cell models, which fail to replicate the complexities of the dynamic mechanical microenvironment. This limitation poses a significant challenge in understanding disease processes, developing diagnostic cancer biomarkers, and identifying therapeutic drug targets for cancer treatment.

Current dynamic biomimetic models (i.e., biomimetic tumor microenvironment models) primarily rely on external devices to drive cyclic tensile stress and observe the behavior and genomic changes of two-dimensional monolayer cells cultured in these dynamic biomimetic models.

Non-small cell lung cancer (NSCLC) exhibits characteristics such as high drug resistance, metastatic potential, low survival rates, and asymptomatic presentation in its early stages, making it difficult to achieve early diagnosis. These characteristics contribute to lung cancer being the leading cause of cancer-related deaths globally. Accordingly, there is an urgent need to investigate the underlying mechanisms driving the malignant progression of NSCLC and to develop anticancer drugs. However, due to the complex physiological structure of the lung, conventional cell culture methods and platforms fail to accurately simulate the physiological conditions of the lung in vivo. Furthermore, preclinical in vitro drug testing predominantly relies on static models, which are insufficient to replicate the complex dynamic mechanical microenvironment observed in vivo.

Additionally, conventional studies investigating the effects of the dynamic mechanical microenvironment on cancer progression primarily focus on monolayer cell cultures, without considering the impact of non-tumor cells (e.g., stromal cells) on cancer progression and drug administration. For example, paracrine growth factors secreted by non-tumor cells may alter the tumor microenvironment, thereby influencing tumor cell growth and the effectiveness of drug treatments. Furthermore, the rigidity of traditional in vitro tumor models cannot be adjusted to closely mimic the tumor microenvironment and is typically limited to altering a single parameter. As a result, such models are insufficient for high-throughput experimental operations and screening. In view of the foregoing, there is an urgent need in the art for a system and method capable of addressing the aforementioned issues by more accurately simulating the dynamic microenvironment of cells and evaluating cell development or the therapeutic efficacy of drugs.

### SUMMARY OF THE INVENTION

A system for mimicking cellular microenvironment, comprising: a chip, a hydrogel, and a cell in contact with the hydrogel. The chip includes a first carrier having a pore and a second carrier coupled to the first carrier. The hydrogel is disposed in the pore and includes an extracellular matrix.

A method for evaluating cell development, including: providing the system for mimicking cellular microenvironment of the disclosure, wherein the hydrogel is a responsive hydrogel; providing a stimulation to the responsive hydrogel to cause a response; and evaluating the cell development.

A method for evaluating drug therapy effectiveness, including: providing the system for mimicking cellular microenvironment of the present disclosure; administering the drug to the cell; and evaluating the drug therapy effectiveness.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a schematic diagram of a chip according to one embodiment of the present disclosure.

FIG. 1B is a schematic diagram of a system for simulating a cellular microenvironment according to one embodiment of the present disclosure.

The upper left panel of FIG. 2 is a photograph of a laser module according to one embodiment of the present disclosure.

The lower left panel of FIG. 2 is a photograph of a laser module according to one embodiment of the present disclosure.

The upper right panel of FIG. 2 is a top and side view of a stand of a laser module according to one embodiment of the present disclosure.

The lower right panel of FIG. 2 is a heat map of a laser module capable of providing a heated environment according to one embodiment of the present disclosure.

FIG. 3A is a flowchart illustrating a system for mimicking cellular microenvironment and method for evaluating cell development or drug therapy effectiveness according to one embodiment of the present disclosure.

FIG. 3B is a bright-field and fluorescent staining image of cells exhibiting malignancy traits according to one embodiment of the present disclosure.

FIG. 3C is a visualization and bar chart showing the malignancy traits of cells treated with inducing factors according to one embodiment of the present disclosure.

FIG. 3D is an immunofluorescent top and side view image of cells exhibiting malignancy characteristics according to one embodiment of the present disclosure.

FIG. 4A is a visualization and bar chart illustrating the effect of hydrogel stiffness on culturing cell spheroids according to one embodiment of the present disclosure.

FIG. 4B is an immunofluorescent image illustrating the effect of hydrogel stiffness on cultured cell spheroids according to one embodiment of the present disclosure.

FIG. 5A is a visualization and bar chart illustrating the effect of hydrogel stiffness on co-cultured cell spheroids according to one embodiment of the present disclosure.

FIG. 5B is an immunofluorescent image illustrating the effect of hydrogel stiffness on co-cultured cell spheroids according to one embodiment of the present disclosure.

FIG. 5C is an immunofluorescent image illustrating the effect of hydrogel stiffness on the self-sorting behavior of co-cultured cell spheroids according to one embodiment of the present disclosure.

FIG. 6 is an immunofluorescent image illustrating the effect of static or dynamic hydrogels on cell spheroids according to one embodiment of the present disclosure.

FIG. 7A is an immunofluorescent image illustrating the effect of static or dynamic hydrogels on cell spheroids according to one embodiment of the present disclosure.

FIG. 7B is an immunofluorescent image illustrating the effect of static hydrogels on two-dimensional monolayer cells according to one embodiment of the present disclosure.

FIG. 8 is an immunofluorescent image illustrating the effect of static or dynamic hydrogels on cell spheroids according to one embodiment of the present disclosure.

FIG. 9 is an immunofluorescent image illustrating the effect of static or dynamic hydrogels on extracellular matrix secretion by cell spheroids according to one embodiment of the present disclosure.

FIG. 10A is a bright-field image illustrating evaluation of the morphological changes of co-cultured cell spheroids treated with drugs according to one embodiment of the present disclosure.

FIG. 10B is a bright-field image illustrating evaluation of the morphological changes of co-cultured cell spheroids treated with drugs according to one embodiment of the present disclosure.

FIG. 10C is a bar chart illustrating evaluation of the morphological changes of co-cultured cell spheroids treated with drugs according to one embodiment of the present disclosure.

FIG. 10D is a bar chart illustrating evaluation of the morphological changes of co-cultured cell spheroids treated with drugs according to one embodiment of the present disclosure.

FIG. 11A is an immunofluorescent image illustrating evaluation of the effect of drug treatment on the viability of co-cultured cell spheroids according to one embodiment of the present disclosure.

FIG. 11B is a line chart illustrating evaluation of the effect of drug treatment on the viability of co-cultured cell spheroids according to one embodiment of the present disclosure.

FIG. 12 is a fluorescent image illustrating evaluation of the effect of drug treatment under static or dynamic stretching condition on the viability of co-cultured cell spheroids according to one embodiment of the present disclosure.

### DETAILED DESCRIPTION

The specific embodiments described below are provided to illustrate the implementation of the present disclosure, enabling a person having ordinary skill in the art to readily understand the spirit, advantages, and efficacy of the disclosure based on the content herein. However, the specific embodiments described herein are not intended to limit the present disclosure. The present disclosure may also be realized or applied through other different embodiments, and the details described herein may be subject to different modifications or adjustments based on various perspectives and applications without departing from the spirit of the disclosure.

The proportions, structures, dimensions, and other features shown in the attached drawings are provided solely to support the description of the present disclosure, aiding persons having ordinary skill in the art in reading and understanding the present disclosure, and are not intended to limit the scope of the implementation of the present disclosure. Therefore, any changes in proportional relationships, structural modifications, or adjustments to dimensions that do not affect the objectives or efficacy achievable by the present disclosure should be considered within the scope disclosed herein.

The terms “comprising,” “including,” or “having” as used herein, unless otherwise stated, may include additional elements, components, structures, regions, parts, devices, systems, steps, modules, or connections, and are not intended to exclude other elements or requirements.

The terms “upper,” “lower,” “before,” and “after” as used herein are solely for the purpose of clarifying specific embodiments of the present disclosure and are not intended to limit the scope of implementation of the present disclosure. Adjustments, substitutions, or alterations to relative positions and relationships that do not materially change the technical content of the present disclosure shall be considered within the scope of the implementation of the present disclosure.

The terms “first” and “second” as used herein are solely for convenience in describing or distinguishing elements, components, structures, regions, parts, devices, systems, or modules and are not intended to limit the scope of the present disclosure, nor are they intended to imply spatial order of those elements. Additionally, unless otherwise explicitly stated, the singular forms “a” and “the” as used herein shall also include the plural form, and the term “or” is interchangeable with “and/or.”

The term “about” as used herein typically refers to a value including the given value or a range with a variance of ±20%, ±10%, ±5%, ±1%, ±0.5%, or ±0.1%. The variance may arise, for example, from experimental error; typical error in measuring, operating, or performing combination, matching, verification, calculation, or correlation processes; the use of different resources, tools, calculation platforms, searches, and matching in the present disclosure; or similar considerations. The term “about” may also refer to values within an acceptable standard error for mean values in the relevant art. Unless explicitly stated otherwise, all numerical ranges, quantities, values, and probabilities described herein, such as those used for the calculation, matching, confidence, execution algorithms, tolerance, and similar factors in present disclosure, shall be understood as being adjustable with the term “about.”

In some embodiments, unless explicitly stated otherwise, the terms “cell” and “cell spheroid” as used herein are interchangeable.

In at least one embodiment of the present disclosure, the system for simulating the cellular microenvironment may include a laser module disposed on the chip to provide a stimulation to the hydrogel. In some embodiments, the laser module provides photostimulation to the hydrogel, thereby causing the hydrogel to respond, such as but not limited to, dynamic stretching, temperature changes, and/or degradation. In some embodiments, the laser module may be disposed on a stand and positioned at a distance from the hydrogel to avoid exposing the hydrogel to excessive laser energy resulting in rapid temperature increases.

In at least one embodiment of the present disclosure, the first carrier may include polydimethylsiloxane (PDMS), but the present disclosure is not limited thereto. In some embodiments, a microfluidic device combined with PDMS material is used to perform stretching, and the behavior and changes in cultured cells are observed.

In at least one embodiment of the present disclosure, the second carrier may include a glass, but the present disclosure is not limited thereto.

In at least one embodiment of the present disclosure, the extracellular matrix may include at least one selected from the group consisting of N-isopropyl acrylamide, gelatin methacrylate, and annealed graphene oxide. In some embodiments, the extracellular matrix may include N-isopropyl acrylamide, gelatin methacrylate, and annealed graphene oxide.

In at least one embodiment of the present disclosure, the cell may include a three-dimensional cell aggregate. In some embodiments, the three-dimensional cell aggregate may be a cell spheroid. In some embodiments, the three-dimensional cell culture exhibits superior tissue specificity compared to two-dimensional cell culture.

In at least one embodiment of the present disclosure, the cell may include at least two types of cells. In some embodiments, the cell may include a tumor cell. In some embodiments, the tumor cell may be a three-dimensional cell aggregate, and the cell may further include a non-tumor cell. In some embodiments, the tumor cell may be a three-dimensional tumor spheroid. In some embodiments, the three-dimensional tumor spheroid may reduce the connection between cells by decomposing the extracellular matrix, and gradually expand and migrate outward, transitioning from a three-dimensional form to a monolayer cell form. This process validates the high invasiveness of the tumor spheroid. Therefore, evaluating the disaggregation capability of tumor spheroids can provide deeper insight into their metastatic potential.

In at least one embodiment of the present disclosure, the hydrogel may be a responsive hydrogel. In some embodiments, a stimulation may be applied to the responsive hydrogel, causing the responsive hydrogel to response.

In at least one embodiment of the present disclosure, the stimulation provided to the hydrogel may include, for example: photostimulation, thermal stimulation, mechanical force stimulation, acid-base stimulation, growth factor stimulation, electric field stimulation, magnetic field stimulation, and/or redox reaction stimulation, but the present disclosure is not limited thereto. In some embodiments, the stimulation applied to the hydrogel may be photostimulation, thermal stimulation, or mechanical force stimulation. In some embodiments, the mechanical force stimulation may be a tensile force stimulation.

In at least one embodiment of the present disclosure, the problems associated with conventional in vitro tumor models are addressed, specifically the lack of investigation into the development of three-dimensional tumor cells in a dynamic mechanical microenvironment and the effects of drug administration. As a result, such conventional in vitro tumor models are unable to replicate the complex dynamic microenvironment of the human body and evaluate the effects of therapeutic drug delivery.

In at least one embodiment of the present disclosure, the evaluation of cell development may include, for example: evaluating cell metastasis capability, cell expansion capability, cell migration capability, the capability of the cell to degrade the extracellular matrix, cell motility, cell disaggregation capability, cell death behavior, cell growth capability, and/or cell viability, but the present disclosure is not limited thereto.

In at least one embodiment of the present disclosure, a factor that induces the malignant characteristics of the cells may be provided.

In at least one embodiment of the present disclosure, the drug therapy effectiveness may include, for example: the capability of cells to absorb the drug, the resistance of cells to the drug, cell metastasis capability, cell expansion capability, cell migration capability, the capability of the cell to degrade the extracellular matrix, cell motility, cell disaggregation capability, cell death behavior, cell growth capability, and/or cell viability, but the present disclosure is not limited thereto.

### EXAMPLES

The following exemplary embodiments are further described in the present disclosure, but they should not be construed as limiting the scope of the present disclosure.

### Materials and Methods

### Preparation of Hydrogel

In some embodiments, the hydrogel of the present disclosure is formed by crosslinking N-isopropyl acrylamide (NIPAM), gelatin methacrylate (GelMA), and annealed graphene oxide (aGO). First, GelMA is dispersed using an ultrasonic oscillator for 30 minutes, followed by the addition of dimethyl sulfoxide (DMSO) in a microcentrifuge tube. Sequentially, 0.0375 wt % aGO solution, 10 wt % NIPAM, 0.5 wt % bis-acrylamide, 0.5 wt % photoinitiator 2-Hydroxy-4′-(2-hydroxyethoxy)-2-methylpropiophenone, and the dispersed GelMA are mixed together. All the components are thoroughly mixed using a vortex mixer. The hydrogel solution is irradiated by ultraviolet (UV) curing lamp (B-100AP lamp, 365 nm, 100 mW) for 10, 20, and 40 minutes, respectively, to form a cured hydrogel. Finally, the cured hydrogel is placed in deionized water overnight to remove excess or unreacted DMSO.

### Preparation of the Chip Containing Polydimethylsiloxane (PDMS) (i.e., the First Carrier as Described in the Present Disclosure)

In some embodiments, the chip of the present disclosure is prepared using DOW CORNING SYLGARD 184 (PDMS) by mixing component A and component B thereof in a ratio of 10:1. The two components are thoroughly mixed until the color turns milky white. The mixture is then placed in a vacuum pump for 30 minutes to remove air bubbles from the solution completely. The degassed PDMS mixture is slowly poured into a mold and again subjected to the vacuum pump for 15 minutes to ensure all air bubbles in the mold are completely removed. Finally, after confirming the mold is bubble-free, the mold is placed in an oven at 60° C. overnight for curing. Once the PDMS is cured, the cured PDMS is removed from the mold and trimmed to the desired size as per user requirements.

### Chip of Assembly of PDMS and Glass Slide (i.e., the Second Carrier as Described in the Present Disclosure)

To achieve a chemical bond between PDMS and the glass slide for tight adhesion, the surfaces of PDMS and the glass slide are exposed to oxygen plasma to introduce numerous hydroxyl groups. The PDMS and glass slide are then pressed tightly together for 30 seconds to 1 minute to form an irreversible chemical bond through siloxane linkages. As a result, the chip composed of the PDMS and the glass slide of the present disclosure can achieve a firm and irreversible bond without using any adhesive. Subsequently, binder clips are used to apply even pressure to all four sides of the chip. After bonding between the PDMS and the glass slide for 3 hours, 3.5 μL of the hydrogel of the present disclosure is added to the pores in the PDMS, followed by irradiation with a UV curing lamp for 20 minutes to solidify the hydrogel. Deionized water is then added on top of the hydrogel to prevent the hydrogel from shrinking.

### Laser Module

In some embodiments, the laser module of the present disclosure is assembled using a relay module and an invisible light laser diode (808 nm, 200 mW). The laser module is set to an infinite cycle mode with the open (OP) and close (CL) times each being 1.5 seconds, thereby simulating the human respiratory rate (0.3 Hz).

### Culture of Human Non-Small Cell Lung Cancer Cells (A549)

In some embodiments, the A549 cell line of the present disclosure is purchased from ATCC, and is commonly used for developing lung cancer drugs and drug delivery experiments. The A549 cells are cultured in F12K cell culture medium (10-025, Corning) supplemented with 10% fetal bovine serum (FBS) (35-076-EV, Corning). The cells are taken out from the liquid nitrogen tank and thawed in a 3° C. water bath. The cell suspension in the cryovial is extracted and mixed with 1 mL of cell culture medium, followed by adding it to a T-75 cell culture flask containing 9 mL of cell culture medium for uniform mixing and culture. When cell confluence reaches 80%, cell passaging is carried out. Cells are first rinsed with DPBS and then detached from the T-75 cell culture flask using trypsin. The detached A549 cells are transferred to a 15 mL centrifuge tube, and centrifuged at 1000 rpm for 5 minutes at 24° C. After centrifugation, the cells are concentrated at the bottom of the centrifuge tube, and the supernatant is aspirated. Subsequently, 500 μL of cell culture medium is added, mixed thoroughly, and the cells are resuspended. A 20 μL cell suspension is then withdrawn, and the cell count is determined using an automatic cell counter, which is used to estimate the total number of cells in the centrifuge tube. The estimated number of cells is then seeded back into the T-75 cell culture flask.

### Culture of Human Lung Fibroblasts (HFL-1)

In some embodiments, the HFL-1 cell line of the present disclosure is purchased from BCRC, and is commonly used for co-culture and cytotoxicity studies. The cells are cultured in F12K cell culture medium (10-025, Corning) supplemented with 10% fetal bovine serum (FBS) (35-076-EV, Corning). The cells are removed from the liquid nitrogen tank and thawed in a 37° C. water bath. The cell suspension in the cryovial is first withdrawn and mixed with 1 mL of cell culture medium and then added to a T-75 cell culture flask containing 9 mL of cell culture medium for even mixing and culture. When the cell confluence reaches 80%, the cells are passaged. The cells are first rinsed with DPBS, and then detached from the T-75 cell culture flask using trypsin. The detached HFL-1 cells are transferred to a 15 mL centrifuge tube, and centrifuged at 1000 rpm for 5 minutes at 24° C. After centrifugation, the cells are concentrated at the bottom of the centrifuge tube. The supernatant is aspirated, and 1 mL of culture medium is added, followed by thorough mixing and resuspension of the cells. A 20 μL cell suspension is then withdrawn, and the cell count is determined using an automatic cell counter to estimate the total number of cells in the centrifuge tube. The estimated number of cells is then seeded back into a T-75 cell culture flask for culture.

### In Vitro 3D Cell Spheroid Culture

In some embodiments, the present disclosure uses Ultra-Low Attachment (ULA) 96-well plates (7007, Corning) to conduct in vitro 3D cell spheroid cultures. The ULA 96-well plates feature an extremely low attachment surface that minimizes cell adhesion and protein absorption, facilitating the formation of cell spheroids. In each well of the ULA 96-well plate, 1000 to 8000 cells (with each well containing 200 μL of cell culture medium) are added, and the morphological changes of the cells are observed. After four days of cell seeding, compact cell spheroids are formed.

### Method for Culturing Cell Spheroids Using the Chip

In some embodiments, before seeding cell spheroids, the chip is irradiated with ultraviolet (UV) light for 30 minutes and washed with filtered 75% alcohol, 95% alcohol, and deionized water, respectively. To enhance cell adhesion, a mixed solution of Type I collagen (collagen I) (CAT #A1048301, Thermo-Gibco) and deionized water (ratio: collagen I:deionized water=1:9) is prepared and added to the chip at 37° C. for 2 hours, and then the excess collagen I solution is removed using DPBS. A 1000 μL pipette tip is aligned with the central depression of the well in the ULA 96-well plate to aspirate the cell spheroids, which are then transferred to a 1.5 μL microcentrifuge tube. The microcentrifuge tube is gently shaken to collect the cell spheroids at the bottom, and the supernatant is aspirated. A 200 μL pipette tip is then used to aspirate the cell spheroids, which are subsequently placed on the hydrogel within the chip. In the cellular microenvironment (i.e., the tumor microenvironment of the present disclosure), transforming growth factor-beta (TGF-β) may induce tumor cell invasion and metastasis, playing a crucial role in tumor progression. Therefore, 20 ng/ml of TGF-β (240-8-002, R&D System) is added to the chip and cultured in an incubator for 15 hours. Subsequently, the laser module is operated in an open (1.5 seconds)/close (1.5 seconds) infinite cycle to deform the hydrogel within the chip, simulating the cyclic tensile mechanical stress experienced during human respiratory movements.

### Staining of Mechanical Response Factors (YAP or F-Actin)

In some embodiments, after the chip is irradiated under the laser module for 6 hours, the cell culture medium on the chip is aspirated, and the pores are washed with DPBS solution. Fixation buffer (Cat #554722, BD Cytofix/Cytoperm™) is then added to fix the cell spheroids on the hydrogel for 15 minutes. The liquid on the hydrogel is aspirated, and the pores are washed again with DPBS. DPBS-diluted mechanical response factor YAP (Cat #14729S, Cell Signaling) (dilution ratio YAP:DPBS=1:200) is added and incubated at room temperature, protected from light for 1.5 hours. The pores are then washed with DPBS twice, and DPBS-diluted F-actin (Cat #424201, Biolegend) (dilution ratio: F-actin:DPBS=1:200) is added and incubated at room temperature, protected from light for 15 minutes. Finally, the hydrogel is washed with DPBS twice, followed by fluorescent staining analysis.

### Staining of Fibronectin of the Extracellular Matrix

In some embodiments, after the chip is irradiated under the laser module for 6 hours, the cell culture medium on the chip is aspirated, and the pores are washed with DPBS solution. Fixation buffer (Cat #554722, BD Cytofix/Cytoperm™) is then added to fix the cell spheroids on the hydrogel for 15 minutes. The liquid on the hydrogel is aspirated, and the pores are washed again with DPBS. DPBS-diluted fibronectin (Cat #ab198934, Abcam) (dilution ratio: Fibronectin:DPBS=1:200) is added and incubated at room temperature, protected from light for 1.5 hours. The pores are then washed with DPBS twice, followed by fluorescent staining analysis.

### Testing of Cell Viability Using CCK-8 Assay (Cell Counting Kit-8)

In some embodiments, the CCK-8 cell viability assay (Enzo Life Sciences, ALX-850-039-KI01) contains WST-1 water-soluble tetrazolium salt, which is reduced by cellular dehydrogenase in the presence of the electron carrier 1-Methoxy PMS, resulting in a highly water-soluble orange formazan dye. The amount of orange formazan produced is directly proportional to the dehydrogenase activity, which reflects the number of viable cells. Therefore, it can be used for the analysis of cell growth and cytotoxicity. To conduct the assay, 100 μL of the culture supernatant from cell spheroids in the ULA 96-well plate is withdrawn, followed by the addition of 10 μL of CCK-8 cell viability reagent to each well. The plate is then incubated at 37° C. for 4 hours. Finally, the ULA 96-well plate is removed from the incubator, and 80 μL of the supernatant from each well is transferred to another 96-well plate. The absorbance at 450 nm is measured using an ELISA reader. The cell viability may be calculated using the following formula:

$$Cell viability ( \% ) = [ ( Drug - treated Group - Blank Group ) / ( Untreated Group - Blank Group ) ] \times 100 \%$$

### Fluorescent Staining of Live/Dead Cells

In some embodiments, the cell viability fluorescent dye (A017, ABP Biosciences) is used for dual-color fluorescent staining of live and dead cells in the cell spheroids to determine cell viability. Initially, 0.1 μL of calcein AM and 0.1 μL of propidium iodide (PI) are added to 1000 μL of DPBS and mixed thoroughly. Subsequently, 100 μL of culture supernatant from the cell spheroids in the ULA 96-well plate is aspirated, and 100 μL of the calcein AM/PI mixture is added to each well. The plate is incubated at 37° C. for 20 minutes. After staining, the ULA 96-well plate is removed from the incubator, and the supernatant of the cell spheroids containing the dye is aspirated. The wells are washed with DPBS three times for five minutes each. Fluorescence microscopy is used to capture the fluorescent images of the stained cell spheroids.

### Fluorescence Staining Analysis

In some embodiments, confocal laser scanning microscopy (LEICA Confocal Laser Scanning Microscope, TCS SP8) is used to observe the adhesion of cell spheroids on the hydrogel, the expression of mechanical response factor, and epithelial-mesenchymal transition events. The hydrogel within the chip are irradiated using lasers with wavelengths of 488 nm and 647 nm, allowing the observation of various phenotypic changes in cell spheroids subjected to laser stimulation.

### Analysis of Cell Spheroid Spreading Area

In some embodiments, Image-J software is used to perform quantitative analysis of the cell spheroid spreading area. The “Color threshold” function is employed to select different fluorescent staining areas, and the “ROI manager” tool is used to create visual representations of the selected areas. The spreading area of cell spheroids is then quantitatively analyzed.

### Results

### Example 1: Establishment of a System for Simulating Cellular Microenvironments

In some embodiments, the hydrogel of the present disclosure is used as the extracellular matrix, and cell spheroids (such as, but not limited to, tumor spheroids) are cultured on the hydrogel. Through the laser module, a photothermal conversion effect is induced to create a dynamic stretching environment, and is further used to evaluate the effects of different mechanical responses on the progression of tumor spheroids and the administration of drugs.

### Construction of a Dynamic Tumor Microenvironment (i.e., Chip 10 of the Present Disclosure)

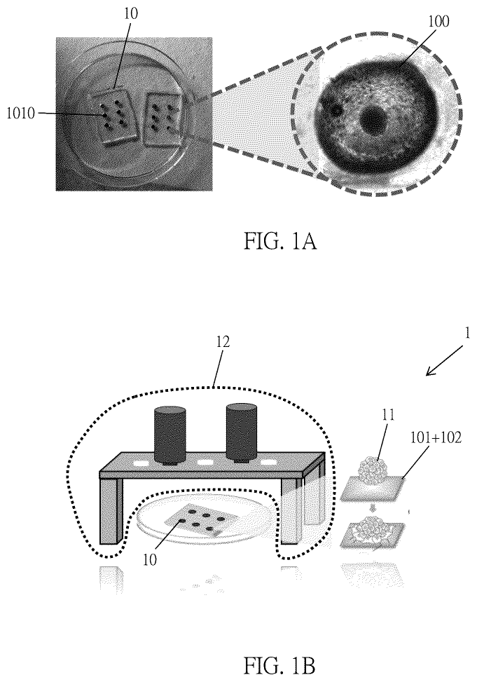

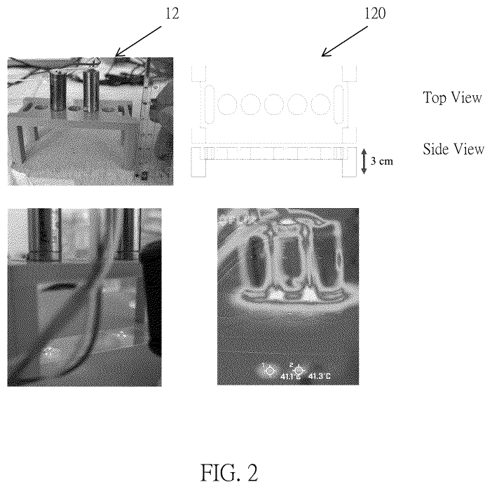

As shown in FIG. 1A, PDMS (i.e., the first carrier of the present disclosure) and a glass slide (i.e., the second carrier of the present disclosure) are covalently bonded through shared electrons, resulting in a stable adhesion. Hydrogel 100 is added into a 3 mm pore 1010 on the PDMS surface, and photo-crosslinking is performed by UV irradiation to form the hydrogel within the pore 1010, thereby completing the construction of the dynamic tumor microenvironment (i.e., chip 10 of the present disclosure).

As shown in FIG. 1B, system 1 for simulating the cellular microenvironment of the present disclosure includes the chip 10, the cells, and the laser module 12. The chip 10 includes the first carrier 101 having the pore 1010, the second carrier 102, and the hydrogel 100 (not shown in FIG. 1B).

### Laser Module 12

As shown in FIG. 2, the laser module 12 is placed on a printed stand 120 (the upper left and upper right panels of FIG. 2). This design effectively fixes the position of the laser lamp. To prevent the hydrogel from being exposed to excessive laser energy leading to rapid temperature increase, the laser lamp is positioned 3 cm away from the hydrogel (the lower left panel of FIG. 2). The lower right panel of FIG. 2 shows the heat map of the laser module providing a heating environment.

### Example 2: Cell Development of Cell Spheroids Cultured on Hydrogels

In some embodiments, cell spheroids or co-cultured cell spheroids are placed on the hydrogel, and the effects of varying matrix stiffness (i.e., the stiffness of the hydrogel) are tested.

### Analysis of the Effects of Growth Factors (e.g., but not Limited to TGF-β Inducing Cell Malignant Characteristics) on the Metastasis of Cell Spheroids

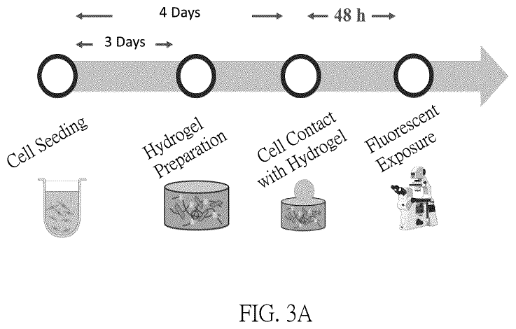

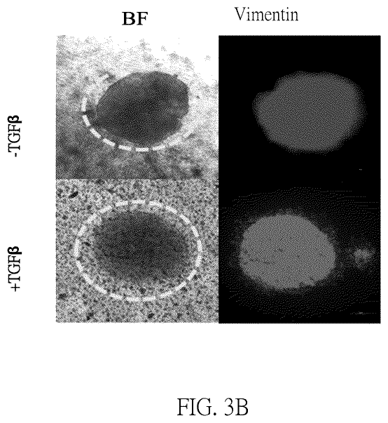

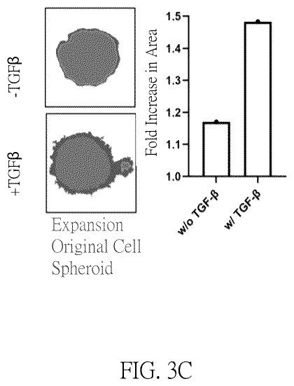

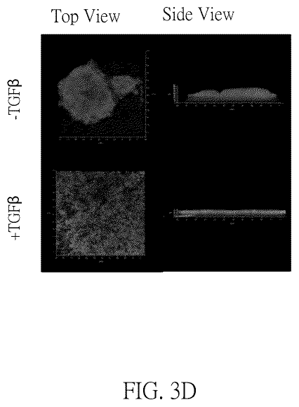

As shown in FIG. 3A, cell spheroids are seeded in a ULA 96-well plate and cultured for four days before being transferred to the hydrogel for further culture. After culturing on the hydrogel for 48 hours, the development of the cell spheroids is observed using a fluorescence microscope. The preparation of the hydrogel is conducted three days after the cell spheroids are seeded. As shown in FIG. 3B, the cell spheroids gradually expanded on the hydrogel after 48 hours of TGF-β addition. The original cell spheroids display red fluorescence indicating the expression of vimentin. FIG. 3C shows the quantification of the cell spheroid spreading area using software, with a visualization clearly indicating the increased outward expansion and migration range of cells in the cell spheroids treated with TGF-β. TGF-β treatment resulted in more malignant characteristics in cells, thereby enhancing cell migration. In FIG. 3C, red (or pink) represents the expansion area, while blue represents the original cell spheroid area. “+TGF-β” (or “w/ TGF-β”) indicates cell spheroids treated with TGF-β, and “−TGF-β” (or “w/o TGF-β”) indicates TGF-β untreated cell spheroids. As shown in FIG. 3D, prolonged TGF-β treatment causes the cell spheroids to disaggregate and expand outward, completely transforming from a three-dimensional structure to a monolayer two-dimensional structure. During this transformation, cells exhibit motile characteristics, such as filopodia preceding migration (which may be indirectly observed by labeling F-actin with green fluorescence). In FIG. 3D, “+TGF-β” indicates TGF-β treatment, while “−TGF-β” indicates no TGF-β treatment. “Top view” and “side view” refer to the respective perspectives, and the green fluorescence labels F-actin.

### Analysis of the Effects of Different Matrix Stiffness (i.e., the Stiffness of the Hydrogel) on Cell Spheroids

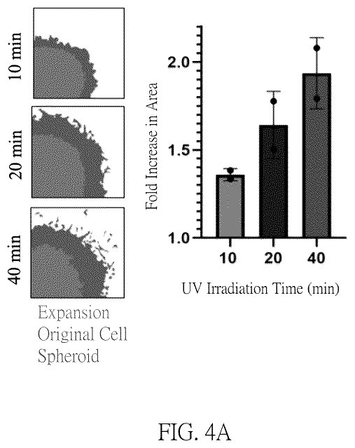

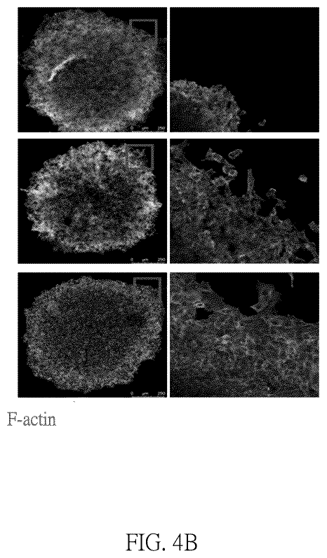

As shown in FIG. 4A, under different curing times (10 min, 20 min, and 40 min) of hydrogel cultivation, the expansion area of cells within A549 cell spheroids increases significantly as the hydrogel stiffness increases. In the group with the longest UV irradiation time (40 min), the cell spheroid expansion area reaches almost twice the original size of the cell spheroid. In FIG. 4A, red (or pink) indicates the expansion area, and blue indicates the original cell spheroid area. As shown in FIG. 4B, the morphology of the cell spheroids is evaluated using fluorescence staining (F-actin labeled for observing the cytoskeleton; green fluorescence). When matrix stiffness increases, the migrating cell spheroids exhibit a polygonal shape, and the area of individual cells also increases correspondingly. In FIG. 4B, the upper panels show the 10 minutes cured condition, the middle panels show the 20 minutes cured condition, and the lower panels show the 40 minutes cured condition. The left panels of FIG. 4B show the view under a low magnification objective lens, while the right panels of FIG. 4B show the view under a high magnification objective lens.

### Analysis of the Effects of Different Matrix Stiffness (i.e., the Stiffness of the Hydrogel) on Co-Cultured Cell Spheroids

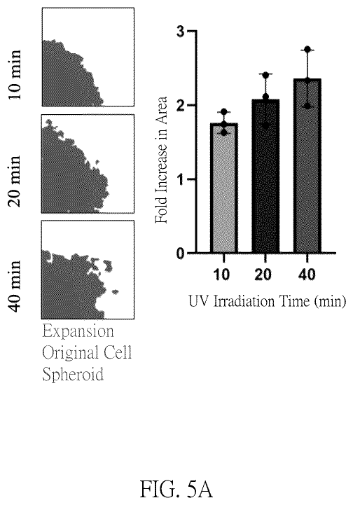

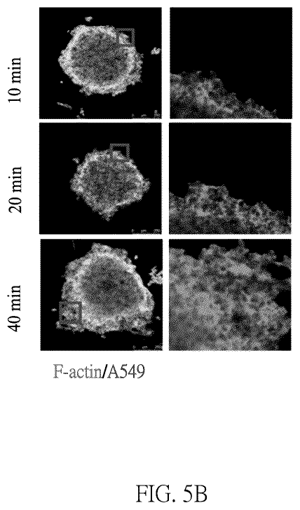

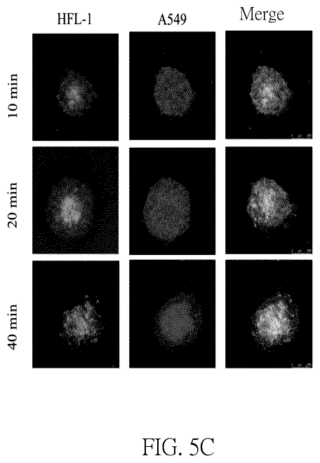

As shown in FIG. 5A, with the increase in matrix stiffness, the expansion area of cells within the cell spheroid also increases accordingly. In FIG. 5A, the red (or pink) color indicates the expansion area, while the blue color represents the original area of the cell spheroid. The labels “10 min,” “20 min,” and “40 min” in FIG. 5A represent the curing times of the matrix using UV radiation for 10 minutes, 20 minutes, and 40 minutes, respectively. As shown in FIG. 5B, with the increase in matrix stiffness, the expression of F-actin (green fluorescence) also significantly increases. In FIG. 5B, the labels “10 min,” “20 min,” and “40 min” indicate curing times of the matrix using UV radiation for 10 minutes, 20 minutes, and 40 minutes, respectively, with red fluorescence marking A549 cells. As shown in FIG. 5C, when co-cultured spheroids are placed on the hydrogel, the co-cultured spheroids exhibit a clear self-sorting effect, allowing indirect observation of the differences in cell behavior between different cell types under mechanical stimulation. In FIG. 5C, the labels “10 min,” “20 min,” and “40 min” represent the curing times of the matrix using UV radiation for 10 minutes, 20 minutes, and 40 minutes, respectively, with green fluorescence marking HFL-1 cells and red fluorescence marking A549 cells. The term “merge” indicates that the left and middle panels of each row in FIG. 5C are combined.

In some embodiments, culturing cell spheroids on the hydrogel of the present disclosure may provide effects different from traditional two-dimensional cell cultures. This allows the observation of cell spheroid metastasis processes that are not visible in traditional two-dimensional cell cultures. Increased matrix stiffness may enhance the migration behavior of the cell spheroids and promote cell self-sorting behavior.

### Example 3: Effects of Culturing Cell Spheroids in a Dynamic Tumor Microenvironment on Cell Spheroid Development

In some embodiments, the hydrogel of the present disclosure is irradiated using a laser module to conduct photothermal conversion for dynamic stretching, and the effects of culturing cell spheroids in a dynamic tumor microenvironment on cell spheroid development are observed.

### Analysis of Mechanical Stimulation Effects on Cell Spheroid Cell Viability

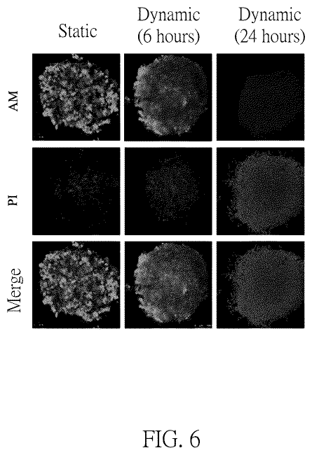

FIG. 6 shows the results of the effects of dynamic stretching of the hydrogel on tumor spheroids. In some embodiments, as shown in FIG. 6, after dynamic stretching of the hydrogel for 6 hours (“Dynamic (6 hours)”), a high and uniform expression of live cells (Calcein AM, abbreviated as AM in FIG. 6; green fluorescence) is observed, with only a small number of dead cells (PI; red fluorescence). This result is consistent with that of the group without laser irradiation (“Static”), confirming that under 6 hours of laser irradiation, cyclic stretching and temperature changes may maintain the viability of the cell spheroids. However, after 24 hours of laser irradiation (“Dynamic (24 hours)”), most cells within the cell spheroids are dead, indicating that the temperature changes induced by laser irradiation may lead to apoptosis of the cells in the cell spheroids.

### Analysis of Mechanical Response Characteristics of the Cell Spheroids

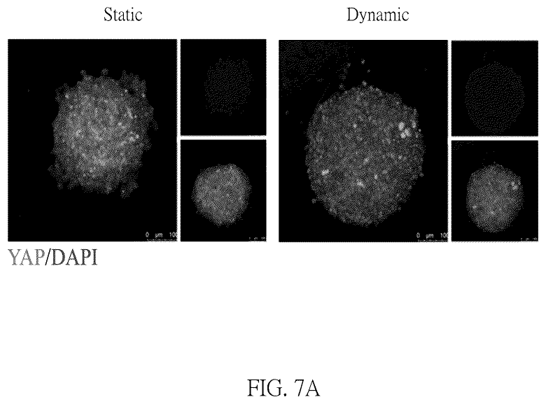

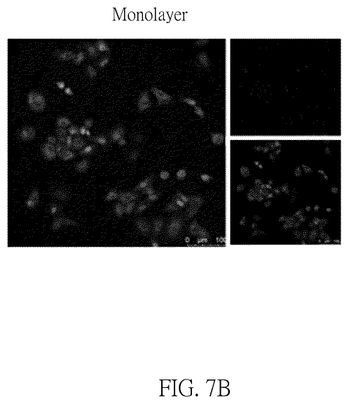

FIGS. 7A and 7B demonstrate the effects of mechanical force on cell spheroids by observing the mechanical response of cells within hydrogels under dynamic stretching conditions. As shown in FIG. 7A, under both static and dynamic conditions, YAP (green fluorescence) is densely expressed in the cytoplasmic region of the cell spheroids, and no translocation to the nucleus (stained with DAPI, blue fluorescence) was observed. In FIG. 7B, A549 cells are seeded at low density and cultured as a two-dimensional monolayer, where the expression of YAP is observed to translocate to the nucleus. The results of FIGS. 7A and 7B indirectly indicate the severity of malignant characteristics in tumor cells.

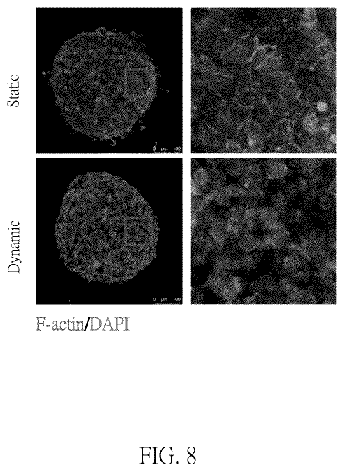

FIG. 8 shows the alignment of the cytoskeleton (F-actin; green fluorescence) of the cell spheroids within the hydrogel under dynamic stretching. As shown in FIG. 8, after 6 hours of dynamic stretching of the hydrogel, the number of cell connections within the cell spheroids is reduced compared to those in static condition. However, no significant rearrangement of the cytoskeleton is observed. DAPI (blue fluorescence) is used to stain the cell nuclei.

### Analysis of the Effect of Mechanical Force on Extracellular Matrix Secretion by Cell Spheroids

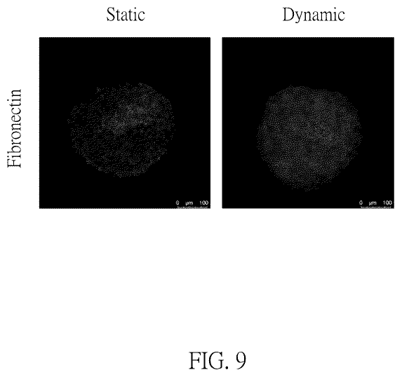

FIG. 9 shows the effect of dynamic stretching of the hydrogel on the secretion of extracellular matrix by cell spheroids. As shown in FIG. 9, the cell spheroids subjected to mechanical stimulation (dynamic condition) expressed more fibronectin (red fluorescence) compared to those not subjected to mechanical stimulation (static condition).

In some embodiments, cell spheroids may be cultured on a dynamic substrate, and it may be observed that mechanical force does not induce corresponding phenotypic changes. However, it is observed that the cytoskeletal arrangement in cells cultured on the dynamic substrate is sparser compared to those under static conditions, and the secretion of extracellular matrix is greater than under static conditions.

### Example 4: Results of Using the System for Simulating the Cellular Microenvironment of the Present Disclosure to Evaluate Drug Therapy Effectiveness

In some embodiments, drugs are applied under static or dynamic conditions to further analyze the effects of dynamic stretching of the hydrogel on drug uptake by cell spheroids.

### Analysis of Drug Resistance in Non-Small Cell Lung Cancer Tumor Spheroids

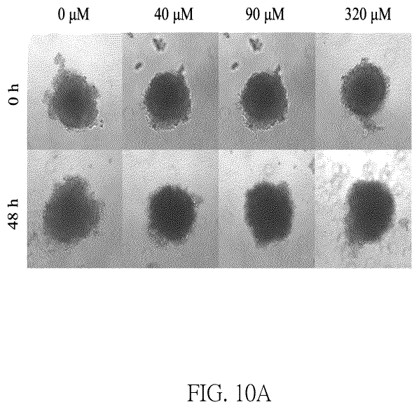

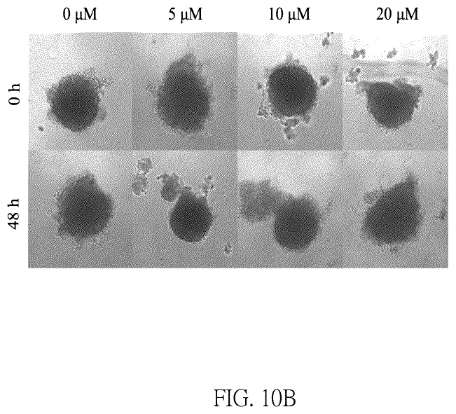

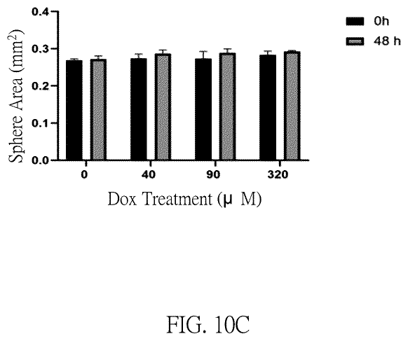

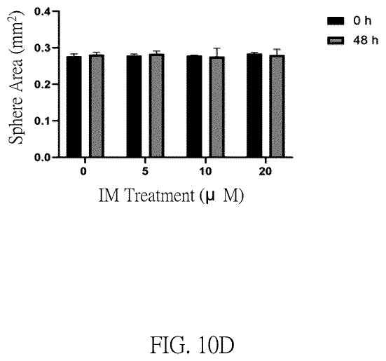

FIGS. 10A and 10B show the results for tumor spheroids after drug administration on the chip. As shown in FIGS. 10A and 10B, cell spheroids treated with different concentrations of the drugs Dox (0 μM, 40 μM, 90 μM, and 320 μM) and IM (0 μM, 5 μM, 10 μM, and 20 μM) maintain their original shape after 48 hours (as indicated by “48 h” in FIG. 10A). Furthermore, as shown in FIGS. 10C and 10D, quantitative analysis of the results in FIGS. 10A and 10B reveal no significant decrease in the area of tumor spheroids.

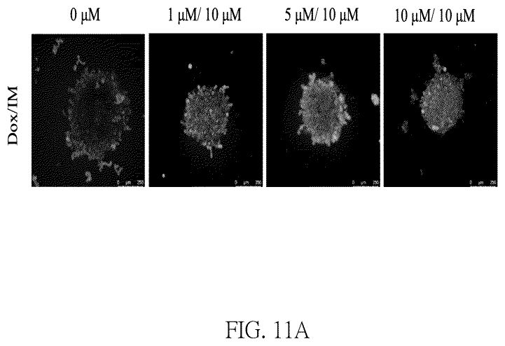

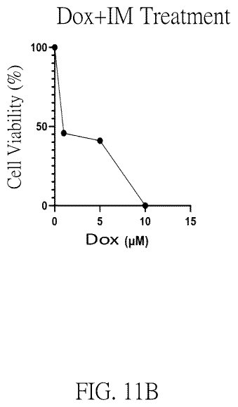

FIGS. 11A and 11B show the results of co-cultured tumor spheroids treated with combination drugs (i.e., a mixture of drugs Dox and IM; indicated as “Dox/IM” in FIG. 11A and “Dox+IM treatment” in FIG. 11B). As shown in FIG. 11A, without the addition of the combination drugs (0 μM Dox/IM), a necrotic area is observed in the center of the cell spheroids. When treated with 10 μM/10 μM Dox/IM, the cell spheroids exhibit red fluorescence overall with a more rounded and smaller shape, indicating that proliferative cells on the periphery have died, causing loose peripheral cells to detach from the cell spheroids. As a result, the cells stop proliferating. However, due to the extracellular matrix structure contained in the cell spheroids, the cell spheroids still maintain an intact spherical shape. In FIG. 11A, red fluorescence indicates dead cells, while green fluorescence indicates live cells. As shown in FIG. 11B, co-cultured tumor spheroids treated with 1 μM/10 μM and 5 μM/10 μM Dox/IM contain more live cells. Cell viability is measured using the CCK-8 cell viability assay showing that at these two concentrations of Dox/IM treatment, the mortality rate of cells in the tumor spheroids is approximately 50%. This confirms that treatment with the combined use of Dox and IM (Dox+IM treatment) effectively increases the drug sensitivity of the cell spheroids, reversing the characteristic of high drug resistance of A549 lung cancer cells.

### Analysis of the Drug Therapy Effectiveness in a Dynamic Tumor Microenvironment

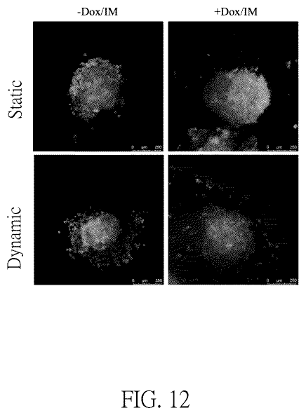

FIG. 12 shows the drug absorption results of co-cultured cell spheroids under dynamic stretching of hydrogel. As shown in FIG. 12, the observation results of cell viability using cell viability fluorescent dyes indicate that, under static conditions, the cell viability of cell spheroids cultured for 6 hours does not significantly differ from that of the group without the drug. However, under dynamic conditions, after 6 hours of cyclic stretching, the drug successfully penetrates the cell spheroids and exerts the effect thereof. Almost all co-cultured cell spheroids exhibit red fluorescence indicating of dead cells. The results show that dynamic cyclic stretching improves the absorption of the drug by cell spheroids, effectively treating tumor cells under the same drug exposure time. In FIG. 11B, red fluorescence indicates dead cells, green fluorescence indicates live cells, +Dox/IM indicates cell spheroids treated with Dox and IM, while −Dox/IM indicates cell spheroids without such Dox and IM treatment.

In some embodiments, drug evaluation under static tumor spheroids finds that IM or Dox alone has no therapeutic effect. However, when drugs are combined (i.e., the combination drug of the present disclosure), it effectively inhibits tumor cells. Upon introduction of dynamic stretching conditions, the drug is found to penetrate the cell spheroids more quickly, thereby achieving therapeutic efficacy.

The above descriptions are merely preferred embodiments of the present invention. Any equivalent changes or modifications made based on the claims of the present disclosure shall fall within the scope of the present disclosure.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A system for mimicking cellular microenvironment, comprising:
a chip, comprising:
a first carrier having a pore; and
a second carrier coupled to the first carrier;
a hydrogel disposed in the pore and comprising an extracellular matrix; and
a cell in contact with the hydrogel.

2. The system of claim 1, further comprising a laser module disposed on the chip to provide a stimulation to the hydrogel.

3. The system of claim 1, wherein the first carrier comprises polydimethylsiloxane, and the second carrier comprises a glass.

4. The system of claim 1, wherein the extracellular matrix comprises at least one selected from the group consisting of N-isopropyl acrylamide, gelatin methacrylate, and annealed graphene oxide.

5. The system of claim 4, wherein the cell comprises a three-dimensional cell aggregate.

6. The system of claim 5, wherein the three-dimensional cell aggregate is a cell spheroid.

7. The system of claim 4, wherein the cell comprises at least two types of cells.

8. The system of claim 7, wherein the cell comprises a tumor cell.

9. The system of claim 8, wherein the tumor cell is a three-dimensional cell aggregate, and the cell further comprises a non-tumor cell.

10. A method for evaluating cell development, comprising:
providing the system of claim 1, wherein the hydrogel is a responsive hydrogel;
providing a stimulation to the responsive hydrogel to cause a response; and
evaluating the cell development.

11. The method of claim 10, wherein the stimulation comprises photostimulation, thermal stimulation, mechanical force stimulation, acid-base stimulation, growth factor stimulation, electric field stimulation, magnetic field stimulation, and/or redox reaction stimulation.

12. The method of claim 11, wherein the system further comprises a laser module disposed on the chip to provide the photostimulation to the responsive hydrogel.

13. The method of claim 10, wherein the cell development comprises metastasis capability, expansion capability, migration capability, capability to degrade the extracellular matrix, motility, disaggregation capability, cell death behavior, cell growth capability, and/or cell viability.

14. A method for evaluating drug therapy effectiveness, comprising:
providing the system of claim 1;
administering the drug to the cell; and
evaluating the drug therapy effectiveness.

15. The method of claim 14, wherein the hydrogel is a responsive hydrogel, and the method further comprises providing a stimulation to the responsive hydrogel to cause a response.

16. The method of claim 15, wherein the stimulation comprises photostimulation, thermal stimulation, mechanical force stimulation, acid-base stimulation, growth factor stimulation, electric field stimulation, magnetic field stimulation, and/or redox reaction stimulation.

17. The method of claim 14, further comprising providing a factor inducing the cell to exhibit malignant characteristics.

18. The method of claim 14, wherein the drug therapy effectiveness comprises the capability to absorb the drug, resistance to the drug, metastasis capability, expansion capability, migration capability, capability to degrade the extracellular matrix, motility, disaggregation capability, cell death behavior, cell growth capability, and/or cell viability.
